(19) United States
(12) Patent Application Publication
(10) Pub. No.: US 2021/0138018 A1
(43) Pub. Date: May 13, 2021

(54) SKIN SENSITIZER DELIVERY SYSTEM

(71) Applicant: Amy Grosso-Piacentino, Culver City, CA (US)

(72) Inventor: Amy Grosso-Piacentino, Culver City, CA (US)

(21) Appl. No.: 17/033,562

(22) Filed: Sep. 25, 2020

Related U.S. Application Data
(63) Continuation of application No. 16/118,301, filed on Aug. 30, 2018, now abandoned.
Application No. 16/118,301 is a continuation of application No. 14/613,308, filed on Feb. 3, 2015, now abandoned.
Application No. 14/613,308 is a continuation of application No. 12/826,069, filed on Jun. 29, 2010, now abandoned.
(60) Provisional application No. 61/221,192, filed on Jun. 29, 2009.

(51) Int. Cl. A61K 36/899; A61K 31/045; A61K 31/122; A61K 31/22; A61K 31/225; A61K 31/343; A61K 31/513; A61K 36/534; A61K 36/54; A61K 36/61; A61K 45/06; A61K 9/00; A61K 9/20; A61K 31/125

(52) U.S. Cl. CPC A61K 36/899; A61K 31/045; A61K 31/122; A61K 31/22; A61K 31/225; A61K 31/343; A61K 31/125; A61K 36/534; A61K 36/54; A61K 36/61; A61K 45/06; A61K 9/0014; A61K 9/2072; A61K 31/513

(57) ABSTRACT

A skin-sensitizing formulation is provided in an edible, softgel pill. A person engaged in a sexual activity bites down on the pill, thereby effectuating the oral release of the skin-sensitizing formulation. This allows the skin-sensitizing formulation to be spread to a sexual partner, on any part of the skin or sexual region of the body preferably through oral contact with the partner. The skin-sensitizing substance is spread to the partner directly on the skin or through oral contact or beneath a condom worn by the partner. The skin-sensitizing formulation may include an essential mint oil, analog or derivative, and may further includes an enhancer, rubefacient, counterirritant, anesthetic, k-opioid receptor activator, or TRPM8 activator. Various inactive ingredients are also disclosed.

### CROSS-REFERENCE TO RELATED APPLICATIONS

This application is a continuation of U.S. patent application Ser. No. 16/118,301, filed Aug. 30, 2018, which is a continuation of U.S. patent application Ser. No. 14/613,308, filed Feb. 3, 2015, which is a continuation of U.S. patent application Ser. No. 12/826,069, filed Jun. 29, 2010, which claims priority to U.S. Provisional Patent Application Ser. No. 61/221,192, filed Jun. 29, 2009, the disclosures of all of which are incorporated herein by reference.

### FIELD OF THE INVENTION

This invention relates generally to skin sensitization and, in particular, to a method of providing a skin-sensitizing formulation within an edible, softgel pill which is bitten by a user to transfer the formulation to a sexual partner through skin contact.

### BACKGROUND OF THE INVENTION

Many couples or individuals want better sensation and pleasure during sex, ways of doing so may be unknown, unsafe, or problematic. Safe sex with condoms can decrease skin sensation for the user, and the receiver, also oral pleasure can be less satisfying for men or women who receive it, if sensation is low in the genital region. Some men find using condoms problematic because of decreased sensation on and in the penis when condoms cover the penis during sexual intercourse. Oral sex can also be less pleasurable to the giver if their mouth lacks sensation or penetration abilities.

### SUMMARY OF THE INVENTION

This invention enhances sexual pleasure, stimulation or satisfaction through a method of delivering a skin-sensitizing substance. According to the method, a skin-sensitizing formulation is provided in an edible, softgel pill. A person engaged in a sexual activity bites down on the pill, thereby effectuating the oral release of the skin-sensitizing formulation. This allows the skin-sensitizing formulation to be spread to a sexual partner, preferably through oral contact with the partner. The skin-sensitizing substance is spread to the partner directly or through oral contact with or under a condom worn by the partner. The composition may also be packaged as an adjunct to use on the interior surface of a condom or alone. The product can be used alone or with a sexual toy.

The skin-sensitizing formulation may include an essential mint oil. For example, the formulation may include mentha piperita, menthol, cinnamon oil, cornmint oil, eucalyptus oil, citronella oil or camphor oil. The formulation may further include an enhancer, rubefacient, countelirritant, anesthetic, k-opioid receptor activator, or Transient receptor potential cation channel subfamily M member 8 (TRPM8) activator. For example, the formulation may include a TRPM8 activator in the form of menthol or icilin.

Alternatively, the skin-sensitizing formulation may include a menthol analog or derivative, such as one or more of the following:

(+)-neo-menthol;

menthone;

(+)-iso-menthone;

menthyl acetate;

menthyl isovalerate;

(−)-menthyl lactate;

para-menth-1-en-3ol;

piperitone;

(−)-menthol ethylene glycol carbonate;

(−)-menthol 1-propylene glycol carbonate;

(−)-menthol 2-propylene glycol carbonate;

(−)-menthone 1,2-glycerol ketal;

(+)-menthone 1,2-glycerol ketal; and

mono-menthyl succinate.

A method of delivering a skin-sensitizing substance in accordance with the invention may comprise the steps of providing an edible, softgel pill containing a skin-sensitizing active ingredient, biting down on the pill to effectuate an oral release of the active ingredient, and spreading the active ingredient to a sexual partner through oral contact; and wherein the active ingredient consists of an essential oil, analog or derivative of an essential oil.

The invention may be used for increased sensation, pleasure, heightened orgasm, awareness, excitement, and sexual thrill. The delivery system allows for more fun during sexual encounters, sensitive sex, increased sensitivity on and inside the penis, increased penetration, deeper throat sensation, deeper kissing, and enhanced sensation of penetration to the vagina, anus, throat, and sensitivity on and in the vagina, as well as increased sensation below a condom. The delivery system can be used on men and on women.

### DETAILED DESCRIPTION OF THE INVENTION

The present invention relates to a new delivery system and method for administering a skin-sensitizing substance by way of a pill. The invention is used by people who have oral (i.e., mouth/tongue/lip) contact with another or themselves, and want escalated, strengthened, sexual pleasure, or higher skin sensation. The term “pill” is used herein for any orally ingestible formulation, with a semi-solid or liquid interior.

According to the invention, a skin sensitizing formulation is suspended in a hermetically sealed, small softgel pill. When the pill is inserted into one's mouth and bitten down on, the formula is promptly released inside the month, and then used from the mouth with saliva and the formula, transferred to the partner's body private region (mouth, penis, vagina, nipples, anus, tongue, or skin). The consumer then can discard the softgel outer shell, or can eat it/swallow it while the delivery system inside works on the partners body/skin, and inside the mouth of the user. Alternatively, the formulation may be packaged as an adjunct to use on the interior surface of a condom or alone, such that upon use of the condom, the formulation is applied topically to the penis, or alone without a condom.

This delivery system allows for increased sensitivity of the skin and genitals. The delivery system is kept inside of a gelatin incasing, that is hermetically sealed, to hold potency and bioavailability until used. The delivery system is quick acting and is small enough to carry around until desired usage. When the pill is broken with the teeth inside the mouth the delivery system begins to take effect inside the mouth and is transferred from the mouth to another, by way of the lips/tongue/saliva/hands from user to the receiver on any body part.

The formulation may contain any sensitizers, enhancers, rubefacients, counterirritants, anesthetics, k-opioid receptor activators, or TRPM8 activators. TRPM8 is an ion channel, upon activation it allows the entry of Na⁺ (sodium) and Ca²⁺ (calcium) ions to the cell that leads to depolarization and the generation of action potential. This eventually leads to the feeling of cold on skin. The TRPM8 protein is expressed in sensory neurons, and it is activated by cold temperatures and cooling agents, such as menthol and icilin where as WS-12 and CPS-369 are the most selective agonist of TRPM8.

According to a preferred embodiment, a gelatin size #3 oval softgel pill is used. The pill has an overall volume of 250 mg with a fill weight of 150 mg. The pill itself is composed of a gelatin shell, with gelatin, purified water and glycerin. The active ingredient is mentha piperita (peppermint essential oil, aerial part). The formulation is preferably low in sodium and free of yeast, wheat, milk or milk derivatives, soy, and artificial colors/flavors.

The formulation may contain other ingredients that are not physiologically active but serve to enhance the pharmaceutical elegance of the final pill or softgel. For example, the compositions of the present invention may contain excipients such as lactose, dextrose, sucrose, sorbitol, mannitol, starch, gum acacia, calcium silicate, microcrystalline cellulose, polyvinylpyrrolidinone, cellulose, water, syrup, and methyl cellulose.

Use of the invention increases sensation, pleasure, heightened orgasm, awareness, excitement, and sexual thrill. This delivery system can be used on men and women. The delivery system allows for more fun, sensitive sex, increased sensitivity on and inside the penis, increased penetration, deeper throat insertion, deeper kissing, and enhanced sensation of penetration to the vagina, anus, throat, and sensitivity on and in the vagina, and increased sensation below a condom before sexual intercourse.

When using this delivery system, men can use condoms and still feel strong sensation on/in the penis while having sexual intercourse, when this delivery system is put on the penis before condom coverage. This can inhibit the spread of contagious diseases such as AIDS and Hepatitis, etc. while still giving heightened pleasure to the user as if the feeling of a condom coverage is not present. Women can also feel the penis better when using the delivery system on/in their genitals, while the penis (with or without condom) is inside the vagina, anus, mouth, or throat.

What is claimed is:

1. A method of delivering a skin-sensitizing substance, comprising the steps of:
providing a skin-sensitizing formulation in an edible, softgel pill;
biting down on the pill to effectuate the oral release of the skin-sensitizing formulation; and
spreading the skin-sensitizing formulation to a sexual partner through oral contact.

2. The method of claim 1, wherein the skin-sensitizing formulation includes an essential mint oil.

3. The method of claim 1, wherein the skin-sensitizing formulation includes mentha piperita.

4. The method of claim 1, wherein the skin-sensitizing formulation includes menthol.

5. The method of claim 1, wherein the skin-sensitizing formulation includes cinnamon oil.

6. The method of claim 1, wherein the skin-sensitizing f01mulation includes cornmint oil.

7. The method of claim 1, wherein the skin-sensitizing formulation includes eucalyptus oil.

8. The method of claim 1, wherein the skin-sensitizing formulation includes citronella oil.

9. The method of claim 1, wherein the skin-sensitizing formulation includes camphor oil.

10. The method of claim 1, wherein the skin-sensitizing formulation further includes an enhancer, rubefacient, counterirritant, anesthetic, K-Opoid Receptor activator, or TRPMS activator.

11. The method of claim 1, wherein the skin-sensitizing formulation further includes a TRPMS activator in the form of menthol or icilin.

12. The method of claim 1, wherein the skin-sensitizing formulation includes a menthol analog or derivative.

13. The method of claim 1, wherein the menthol analog or derivative includes one or more of the following:
(+)-neo-menthol;
menthone;
(+)-iso-menthone;
menthyl acetate;
menthyl isovalerate;
(−)-menthyl lactate;
para-menth-1-en-3ol;
piperitone;
(−)-menthol ethylene glycol carbonate;
(−)-menthol 1-and 2-propylene glycol carbonate;
(−)-menthone 1,2-glycerol ketal;
(+)-menthone 1,2-glycerol ketal; and
mono-menthyl succinate.

14. The method of claim 1, wherein the skin-sensitizing substance is spread to the partner through oral contact, or under a condom, worn by the partner.

15. A method of delivering a skin-sensitizing substance, comprising the steps of:
providing an edible, softgel pill containing a skin-sensitizing active ingredient;
biting down on the pill to effectuate an oral release of the active ingredient;
spreading the active ingredient to a sexual partner through oral contact; and
wherein the active ingredient consists of an essential oil or an analog or derivative or an essential oil.

16. The method of claim 15, wherein the active ingredient is spread to the partner primarily through oral contact with a woman or a man, or below a condom worn by the partner.
